(19) United States
(12) Patent Application Publication
(10) Pub. No.: US 2026/0215753 A1
(43) Pub. Date: Jul. 30, 2026

(54) SYSTEMS AND METHODS FOR ASSESSING PATIENT CONDITIONS BASED ON CARDIOVASCULAR SOUNDS

(71) Applicant: Medtronic, Inc., Minneapolis, MN (US)

(72) Inventors: Todd M. Zielinski, McGrath, MN (US); Neethu Lekshmi Vasudevan Jalaja, Manor, TX (US); Anna J. Malin, Paradise Valley, AZ (US); Shantanu Sarkar, Roseville, MN (US); Paul G. Krause, Mahtomedi, MN (US); Catherine R. Condie, Shoreview, MN (US)

(21) Appl. No.: 19/451,224

(22) Filed: Jan. 16, 2026

Related U.S. Application Data
(60) Provisional application No. 63/750,149, filed on Jan. 27, 2025.

(51) Int. Cl. A61B 7/04; A61B 5/00; A61B 7/00; A61B 7/02

(52) U.S. Cl. CPC A61B 7/04; A61B 5/4842; A61B 5/686; A61B 7/005; A61B 7/023; A61B 2560/0214; A61B 2560/0468; A61B 2562/0204; A61B 2562/0219

(57) ABSTRACT

Systems and methods for assessing patient conditions based on cardiovascular sounds are disclosed herein. According to some variations, a system includes a sensor arrangement implantable in a patient and configured to provide a sensor signal indicative of heart sounds and/or other cardiovascular sounds of the patient, and a processor configured to receive the sensor signal and characterize a heart valve condition and/or peripheral vascular condition of the patient by using an artificial intelligence model to analyze the sensor signal.

### RELATED APPLICATIONS

This application claims the benefit of U.S. Provisional Application Ser. No. 63/750,149, filed Jan. 27, 2025, the entire contents of each of which are incorporated herein by reference.

### TECHNICAL FIELD

The present technology relates to systems and methods for assessing patient conditions based on cardiovascular sounds.

### BACKGROUND

Many medical conditions in a patient may be difficult to detect, which may leave afflicted patients in an undesirable state in which progression of the undiagnosed condition may lead to patient harm, including death. For example, some conditions such as aortic abdominal aneurysms are completely silent in that they are not accompanied by pain or any other readily noticeable symptoms, but early detection of such conditions is often important to facilitate timely medical intervention. Early detection may sometimes be achieved through regular examinations and/or imaging, but this not always practicable or possible for patients.

### SUMMARY

The subject technology is illustrated, for example, according to various aspects described below, including with reference to FIGS. 1-8. Various examples of aspects of the subject technology are described as numbered clauses (1, 2, 3, etc.) for convenience. These are provided as examples and do not limit the subject technology.
1. A system comprising:
   a sensor arrangement implantable in a patient and configured to provide a sensor signal indicative of heart sounds of the patient;
   a processor; and
   a memory operably coupled to the processor and storing instructions that, when executed by the processor, cause the system to perform operations comprising:
      receiving the sensor signal; and
      characterizing a heart valve condition of the patient by using an artificial intelligence model to analyze the sensor signal.
2. The system of clause 1, wherein the heart sounds comprise at least one of S1, S2, S3, and S4 heart sounds.
3. The system of clause 1 or 2, wherein the operations further comprise extracting one or more features of the sensor signal including at least one of frequency, amplitude, or energy over time.
4. The system of any one of clauses 1-3, wherein the artificial intelligence model comprises a pre-trained machine learning model.
5. The system of clause 4, wherein the pre-trained machine learning model is at least partially trained on data personalized to the patient.
6. The system of any one of clauses 1-5, wherein characterizing the heart valve condition comprises evaluating at least one of presence of the heart valve condition or progression of the heart valve condition over time.
7. The system of any one of clauses 1-6, wherein the sensor arrangement comprises at least one of an accelerometer, a microphone, or a piezoelectric crystal.
8. The system of any one of clauses 1-7, wherein the heart valve condition comprises at least one of stenosis or regurgitation of an aortic valve in the patient.
9. The system of any one of clauses 1-8, wherein the heart valve condition comprises at least one stenosis or regurgitation of a mitral valve in the patient.
10. The system of any one of clauses 1-9, wherein the heart valve condition comprises a status of an artificial heart valve implanted in the patient.
11. The system of any one of clauses 1-10, wherein the sensor arrangement, the processor, and the memory are incorporated in an implantable device.
12. The system of clause 11, wherein the implantable device comprises an insertable cardiac monitor comprising a power source and one or more electrodes.
13. The system of clause 12, wherein the insertable cardiac monitor is configured to be implanted subcutaneously in the patient.
14. The system of clause 12 or 13, wherein the insertable cardiac monitor has a volume of about 1.5 cubic centimeters or less.
15. A computer-implemented method, comprising:
   receiving a sensor signal from a sensor arrangement implanted in a patient, wherein the sensor signal is indicative of heart sounds of the patient; and
   characterizing a heart valve condition of the patient by using an artificial intelligence model to analyze one or more features of the sensor signal, wherein the one or more features comprises at least one of frequency, amplitude, or energy over time.
16. A system comprising:
   a sensor arrangement implantable in a patient and configured to provide a sensor signal indicative of cardiovascular sounds of the patient;
   a processor; and
   a memory operably coupled to the processor and storing instructions that, when executed by the processor, cause the system to perform operations comprising:
      receiving the sensor signal; and
      characterizing a peripheral vasculature condition of the patient by using an artificial intelligence model to analyze the sensor signal.
17. The system of clause 16, wherein the operations further comprise extracting one or more features of the sensor signal including at least one of frequency, amplitude, or energy over time.
18. The system of clause 16 or 17, wherein the artificial intelligence model comprises a pre-trained machine learning model.
19. The system of clause 18, wherein the pre-trained machine learning model is at least partially trained on data personalized to the patient.
20. The system of any one of clauses 16-19, wherein the peripheral vasculature condition comprises at least one of an abdominal aortic aneurysm, deep vein thrombosis, hemorrhage, occlusion, or peripheral vascular disease.
21. The system of any one of clauses 16-20, wherein the sensor arrangement, the processor, and the memory are incorporated in an implantable device.
22. The system of any one of clauses 16-21, wherein the implantable device comprises an insertable cardiac monitor comprising a power source and one or more electrodes.
23. The system of any one of clauses 16-22, wherein the insertable cardiac monitor is configured to be implanted subcutaneously in the patient.
24. The system of any one of clauses 16-23, wherein the insertable cardiac monitor has a volume of about 1.5 cubic centimeters or less.
25. A computer-implemented method, comprising:
   receiving a sensor signal from a sensor arrangement implanted in a patient, wherein the sensor signal is indicative of cardiovascular sounds of the patient; and
   characterizing a condition of peripheral vasculature of the patient by using an artificial intelligence model to analyze one or more features of the sensor signal, wherein the one or more features comprises at least one of frequency, amplitude, or energy over time.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is an illustrative flowchart of an example method for assessing patient conditions based on cardiovascular sounds, in accordance with the present technology.

FIG. 2 is an illustrative flowchart of an example method for assessing patient conditions based on cardiovascular sounds, in accordance with the present technology.

FIG. 3A is an illustrative schematic of an example variation of an insertable cardiac monitoring device, in accordance with the present technology.

FIG. 3B is a conceptual diagram of an example variation of an insertable cardiac monitoring device, in accordance with the present technology.

FIG. 4 is an illustrative schematic of a cardiac monitoring system including an insertable cardiac monitoring device placed in a patient, in accordance with the present technology.

FIG. 5 is an illustrative schematic of an example variation of an insertable cardiac monitoring device, in accordance with the present technology.

FIG. 6 is an illustrative flowchart of an example method for assessing valvular dysfunction based on heart sounds, in accordance with the present technology.

FIG. 7 is a diagram illustrating example heart murmurs in relation to heart sounds and heart cycle phases.

FIG. 8 is an illustrative flowchart of an example method for assessing peripheral vasculature conditions based on cardiovascular sounds, in accordance with the present technology.

### DETAILED DESCRIPTION

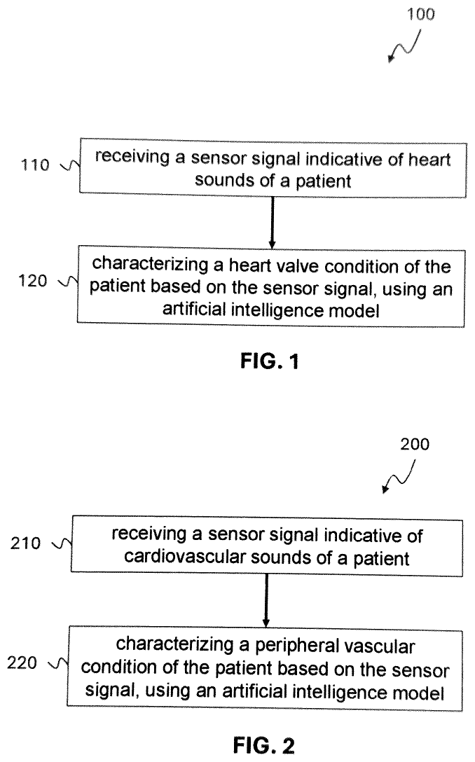

The present technology relates to systems and methods for assessing patient conditions based on cardiovascular sounds. Some variations of the present technology, for example, are directed to detecting heart valve conditions based on an analysis of heart sounds. As another example, some variations of the present technology are directed to detecting peripheral vascular conditions based on an analysis of cardiovascular sounds. Specific details of several variations of the technology are described below with reference to FIGS. 1-8.

Generally, as described in further detail herein, various patient conditions may be assessed using an implantable device (e.g., insertable cardiac monitor, or other medical device with a suitable sensor arrangement) having a sensor arrangement configured to detect sounds, such as heart sounds or other cardiovascular sounds. Computer-implemented methods may analyze the sensor signal(s) indicative of such cardiovascular sounds of a patient, to detect or otherwise characterize a medical condition of the patient. In some variations, because the sensor arrangement is implanted in the patient and may be continuously recording and/or analyzing sounds associated with the patient, the implanted device with the sensor arrangement may facilitate continuous patent monitoring. This in turn can facilitate early detection of certain medical conditions and/or better tracking of progression of medical conditions, thereby enabling timely medical treatment or other intervention and leading to better patient outcomes.

For example, FIG. 1 is a schematic flowchart of an example method 100 for assessing a patient condition based on sounds. The method 100 includes receiving a sensor signal indicative of heart sounds of a patient 110, and characterizing a heart valve condition of the patient based on the sensor signal 120, such as by use of an artificial intelligence model (e.g., machine learning model). In some variations, the sensor signal is obtained with a sensor arrangement including one or more heart sound sensors (e.g., accelerometer, microphone, piezoelectric crystal element) and/or other concurrent sensors, where the sensor arrangement is part of an implantable device. The implantable device may include multiple functionalities. For example, in some variations the implantable device may be a cardiac monitoring device including a sensor arrangement configured to measure electrical activity of the heart, as well as detect heart sounds. However, in some variations the implantable device may include a sensor arrangement configured to detect solely heart sounds. Furthermore, in some variations at least a portion of the sensor arrangement may be arranged in an external device, such as a wearable device.

As another example, FIG. 2 is a schematic flowchart of an example method 200 for assessing a patient condition based on sounds. The method 200 includes receiving a sensor signal indicative of cardiovascular sounds of a patient 210, and characterizing a peripheral vascular condition of the patient based on the sensor signal 220, such as by use of an artificial intelligence model (e.g., machine learning model). In some variations, the sensor signal is obtained with a sensor arrangement including one or more cardiovascular sound sensors (e.g., accelerometer, microphone, piezoelectric crystal element) and/or other concurrent sensors, where the sensor arrangement is part of an implantable device. The implantable device may include multiple functionalities. For example, in some variations the implantable device may be a cardiac monitoring device including a sensor arrangement configured to measure electrical activity of the heart, as well as detect cardiovascular sounds. However, in some variations the implantable device may include a sensor arrangement configured to detect solely cardiovascular sounds. The implantable device can, in some variations, be configured to be placed proximate an anatomical region of interest, such as the torso for detecting abdominal aortic aneurysms. Furthermore, in some variations at least a portion of the sensor arrangement may be arranged in an external device, such as a wearable device.

### I. Monitoring Devices and Systems

As described above, in some variations an implantable monitoring device may be configured to perform at least a portion of the method 100 and/or 200 for assessing patient conditions. Described below in detail are examples of implantable monitoring devices that are insertable cardiac monitors. However, it should be understood that the methods 100 and 200 may performed at least in part by any suitable implantable device and/or external device (e.g., wearable device).

Cardiac monitoring devices are used to measure electrical activity of the heart and record cardiac information of a patient such as heart rate and rhythm. The cardiac electrical activity data is primarily referred to herein as electrogram (EGM) data, although for the methods and systems described herein the cardiac electrical activity may additionally or alternatively include electrocardiogram (ECG) or other signal data. The EGM data can be analyzed, for example, to detect the occurrence of cardiac events experienced by the patient, and may be used by clinicians to diagnose cardiac disorders (e.g., cardiac rhythm disorders).

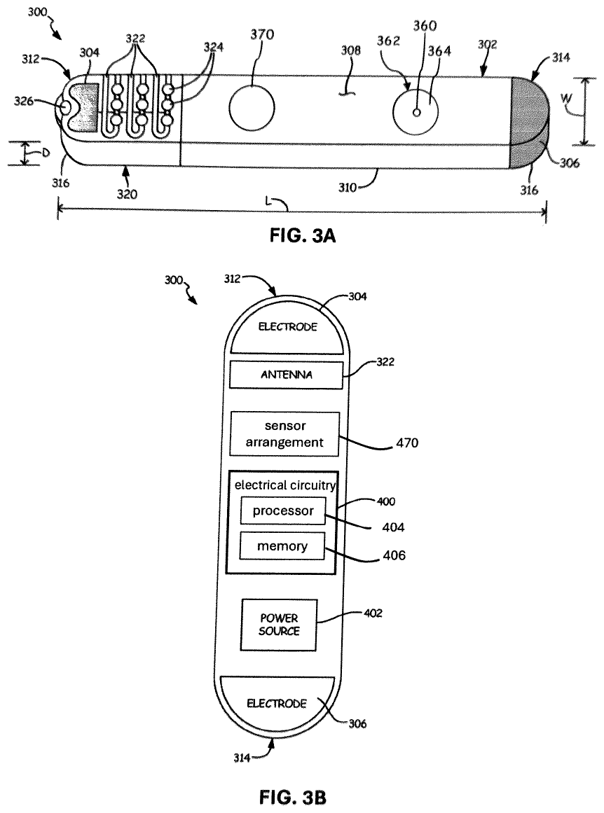

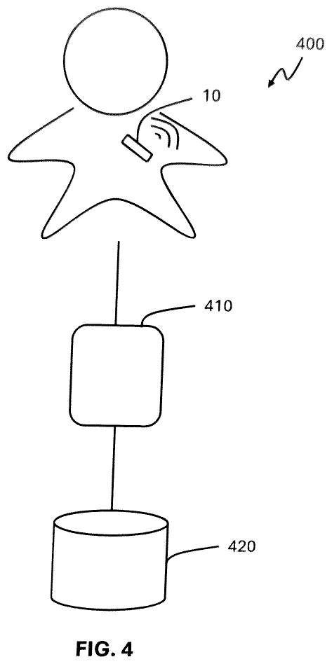

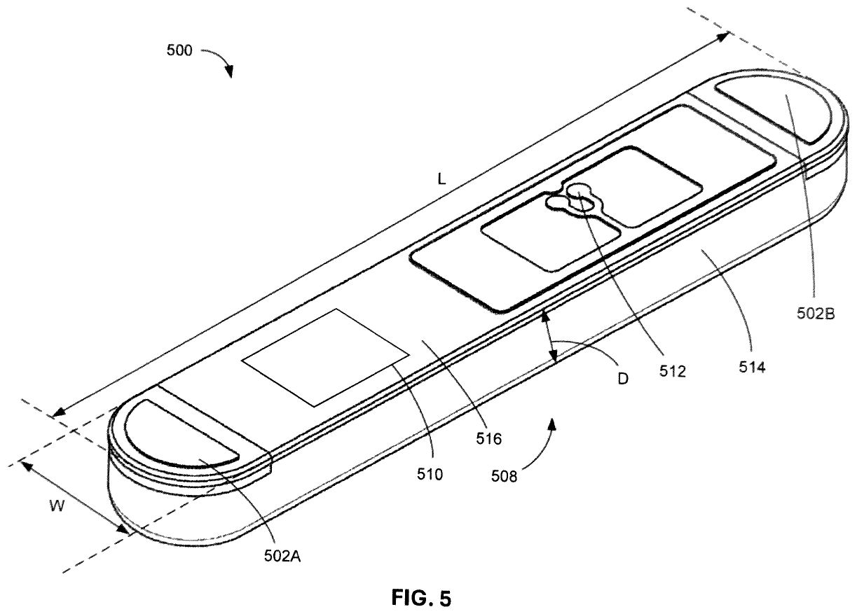

FIG. 3A is a conceptual diagram of an example of an insertable cardiac monitor (ICM) 300 (also referred to herein as a “cardiac monitoring device”) for detecting a bradycardia/asystole event, according to another variation of the present disclosure. In the example shown in FIG. 3A, insertable cardiac monitor 300 may be embodied as a monitoring device having housing 302, a first (e.g., proximal) electrode 304, and a second (e.g., distal) electrode 306. Housing 302 may further comprise a first major surface 308, a second major surface 310, a first (e.g., proximal) end 312, and a second (e.g., distal) end 314. Housing 302 encloses electronic circuitry 400 and power source 402 (shown in FIG. 4) located inside the insertable cardiac monitor 300 and protects the circuitry contained therein from body fluids. Electrical feedthroughs provide electrical connection of electrodes 304 and 306.

In some variations such as that shown in FIG. 3A, insertable cardiac monitor 300 is defined by a length L, a width W and thickness or depth D. The ICM may be in the form of an elongated rectangular prism wherein the length L is much larger than the width W, which in turn is larger than the depth D. In some variations, the geometry of the insertable cardiac monitor 300 (for example, a width W greater than the depth D) may be selected to allow the cardiac monitor 300 to be inserted under the skin of the patient using a minimally invasive procedure and to remain in the desired orientation during insert. For example, the device shown in FIG. 3A may include radial asymmetries (notably, the rectangular shape) along the longitudinal axis that maintains the device in the proper orientation following insertion. For example, in some variations the spacing between the proximal electrode 304 and distal electrode 306 may range from 30 millimeters (mm) to 55 mm, 35 mm to 55 mm, and from 40 mm to 55 mm and may be any range or individual spacing from 25 mm to 60 mm. In addition, insertable cardiac monitor 300 may have a length L that ranges from 30 mm to about 70 mm. In other variations, the length L may range from 40 mm to 60 mm, 45 mm to 60 mm and may be any length or range of lengths between about 30 mm and about 70 mm. In addition, the width W of major surface 308 may range from 3 mm to 10 mm and may be any single or range of widths between 3 mm and 10 mm. In some variations, the thickness of depth D of the insertable cardiac monitor 300 may range from 2 mm to 9 mm. For example, the depth D of the insertable cardiac monitor 300 may range from 2 mm to 5 mm and may be any single or range of depths from 2 mm to 9 mm. In addition, insertable cardiac monitor 300 according to an example variation of the present invention has a geometry and size designed for ease of implant and patient comfort. Variations of insertable cardiac monitor 300 described in this disclosure may have a volume of three cubic centimeters (cm) or less, 1.5 cubic cm or less or any volume between three and 1.5 cubic centimeters.

In the example shown in FIG. 3A, once inserted within the patient, the first major surface 308 faces outward, toward the skin of the patient while the second major surface 310 is located opposite the first major surface 308. In addition, in the example shown in FIG. 3A, proximal end 312 and distal end 314 are rounded to reduce discomfort and irritation to surrounding tissue once inserted under the skin of the patient. Insertable cardiac monitor 300, including instrument and method for inserting monitor 300 is described, for example, in U.S. Patent Publication No. 2014/0276928, incorporated herein by reference in its entirety.

As described with other variations, proximal electrode 304 and distal electrode 306 may be used to sense cardiac signals for determining a cardiac event (e.g., bradycardia or asystole event) such as EGM signals, intra-thoracically or extra-thoracically, which may be sub-muscularly or subcutaneously. EGM signals may be stored in a memory of the insertable cardiac monitor 300, and EGM data may be transmitted via integrated antenna 322 to another medical device, which may be another implantable device or an external device.

In the example variation shown in FIG. 3A, proximal electrode 304 is in close proximity to the proximal end 312 and distal electrode 306 is in close proximity to distal end 314. In this variation, distal electrode 306 is not limited to a flattened, outward facing surface, but may extend from first major surface 308 around rounded edges 316 and onto the second major surface 310 so that the electrode 306 has a three-dimensional curved configuration. In the example variation shown in FIG. 3A, proximal electrode 304 is located on first major surface 308 and is substantially flat, outward facing. However, in other variations, proximal electrode 304 may utilize the three-dimensional curved configuration similar to that of distal electrode 306, providing a three-dimensional proximal electrode (not shown in this variation). Additionally or alternatively, in other variations, distal electrode 306 may utilize a substantially flat, outward facing electrode located on first major surface 308 similar to that shown with respect to proximal electrode 304. The various electrode configurations allow for configurations in which proximal electrode 304 and distal electrode 306 are located on both first major surface 308 and second major surface 310. In other configurations, such as that shown in FIG. 3A, only one of proximal electrode 304 and distal electrode 306 is located on both major surfaces 308 and 310, and in still other configurations both proximal electrode 304 and distal electrode 306 are located on one of the first major surface 308 or the second major surface 310 (e.g., proximal electrode 304 located on first major surface 308 while distal electrode 306 is located on second major surface 310). In some variations, the insertable cardiac monitor 300 may include electrodes on both major surface 308 and 310 at or near the proximal and distal ends of the device, such that a total of at least four electrodes are included on cardiac monitor device 300. Electrodes 304 and 306 may be formed of a plurality of different types of biocompatible conductive material (e.g. stainless steel, titanium, platinum, iridium, or alloys thereof), and/or may utilize one or more coatings such as titanium nitride or fractal titanium nitride.

In the example shown in FIG. 3A, proximal end 312 includes a header assembly 320 that includes one or more of proximal electrode 304, an integrated antenna 322, anti-migration projections 324, and/or suture hole 326. The integrated antenna 322 may be located on the same major surface (e.g., first major surface 308) as proximal electrode 304 and may also be included as part of header assembly 320. Integrated antenna 322 allows insertable cardiac monitor 300 to transmit and/or receive data. In some variations, integrated antenna 322 may be formed on the opposite major surface as proximal electrode 304, or may be incorporated within the housing 322 of insertable cardiac monitor 300. In the example variation shown in FIG. 3A, anti-migration projections 324 are located adjacent to integrated antenna 322 and protrude away from first major surface 308 to prevent longitudinal movement of the device, though may be arranged on any suitable surface of the insertable cardiac monitor 300. In the example variation shown in FIG. 3A, anti-migration projections 324 include a plurality (e.g., nine) small bumps or protrusions extending away from first major surface 308; however, anti-migration projections 324 may additionally or alternatively be located on the opposite major surface as proximal electrode 304 and/or integrated antenna 322. As shown in FIG. 3A, the suture hole 326, which may be used to help secure the insertable cardiac monitor 300 in the patient to prevent movement following insertion of the insertable cardiac monitor 300, may be located adjacent to proximal electrode 304, though one or more suture holes 326 may additionally or alternatively be located in any other suitable location. In some variations, the header assembly 320 is a molded header assembly made from a polymeric or plastic material, which may be integrated or separable from the main portion of insertable cardiac monitor 300.

In some variations, the device 300 may include an optical sensor arrangement configured to detect light. The optical sensor arrangement may be under an optically transparent region enabling pass through of light to the optical sensor arrangement. For example, in some variations, a portion of the first major surface 308 is optically transparent to enable pass through of optical signals such as light into and out of the device 300 while housing 302 is hermetically sealed. The portion of the first major surface 308 which is optically transparent may comprise an aperture 362 with an optical window 364 that allows at least a portion of a visible light spectrum and/or a near-visible light spectrum (e.g., infrared light or ultraviolet light) to pass through to an optical sensor arrangement 360. In some examples, the optically transparent region (e.g., window) comprises an optically clear material such as sapphire, glass, and/or plastic. In some examples, the optical window 364 may be formed of the same material as the housing 302 and/or the first major surface 308. Additionally or alternatively, the second major surface 310 can comprise an optical window (e.g., the optical window 364). Though shown as having a circular area, the aperture 362 of the optical window 364 may be of any shape (e.g., triangular, rectangular, polygonal, curvilinear). In some examples, the optical window 364 comprises the entire first major surface 308 and/or other surface of the housing 302. In some examples, the first major surface 308 and/or any other suitable surface of the device 300 is optically transparent (e.g., the first major surface 308 comprises transparent sapphire).

In some variations, the device 300 may additionally or alternatively include a sensor arrangement 370 including sensors other than optical sensors. For example, the sensor arrangement 370 may include one or more sensors configured to detect and/or measure sounds (e.g., accelerometer, microphone, piezoelectric crystal element). When the insertable cardiac monitor is placed proximate the heart, for example, the sensor arrangement may be configured to detect heart sounds (e.g., S1, S2, S3, S4, etc.), though the sensor arrangement may be configured to detect other cardiovascular sounds and/or other types of body sounds when the monitoring device is placed proximate other anatomical regions of interest. Additionally or alternatively, in some variations, the sensor arrangement 370 may include other kinds of sensors enabling concurrent sensing of additional physiological parameters, such as an activity sensor, a pressure sensor, an oxygen sensor, temperature sensor, and/or an impedance sensor.

FIG. 3B is a functional schematic diagram of the insertable cardiac monitor 300 as shown in FIG. 3A in accordance with the present technology. Insertable cardiac monitor 300 includes housing 302, proximal electrode 304 located at proximal end 312, distal electrode 306 located at distal end 314, integrated antenna 322, electrical circuitry 400 and power source 402. Electrical circuitry 400 may be coupled to proximal electrode 304 and distal electrode 306 to sense cardiac signals and monitor events (e.g., arrythmia, etc.). Electrical circuitry 400 is also connected to transmit and receive communications via integrated antenna 322. Power source 402 provides power to electrical circuitry 400, as well as to any other components that require power. Power source 402 may include one or more energy storage devices, such as one or more rechargeable or non-rechargeable batteries. The insertable cardiac monitor 300 as shown in FIGS. 3 and 4 may be a monitoring-only device. However, in other examples, insertable cardiac monitor 300 may further provide therapy delivery capabilities.

The electrical circuitry 400 receives raw EGM signals monitored by proximal electrode 304 and distal electrode 306. Electrical circuitry 400 may also include components/modules for converting the raw EGM signal to a processed EGM signal that can be analyzed to detect sense events. Although not shown, electrical circuitry 400 may include any discrete and/or integrated electronic circuit components that implement analog and/or digital circuits capable of producing the functions described for analyzing EGM signals to detect/verify bradycardia and/or asystole events. For example, the electrical circuitry 400 may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, one or more processors 404 (shared, dedicated, or group) that executes one or more software or firmware programs, memory devices 406, or any other suitable components or combination thereof that provide the described functionality.

In some variations, electrical circuitry 400 may include a sensing unit for monitoring the EGM signal detected by the respective proximal and distal electrodes 304 and 306, respectively, and at least one sensing channel that utilizes an algorithm for identifying events in the EGM signal. For example, sensed events (e.g., R-waves) are utilized to detect one or more cardiac episodes. In some variations, electrical circuitry 400 includes a processor is utilized to receive information regarding the sensed events and implements one or more algorithms for determining whether a particular one or more events have occurred. In addition, the analog voltage signals received from electrodes 304 and 306 may be passed to analog-to-digital (A/D) converters included in the electrical circuitry 400, and stored in a memory unit 406 included as part of electrical circuitry 400 for subsequent analysis with firmware executed by the processor(s) 404 included as part of electrical circuitry 400.

Electrical circuitry 400 may control insertable cardiac monitor 300 functions and process EGM signals received from electrodes 304 and 306 according to programmed signal analysis routines or algorithms. The insertable cardiac monitor 300 may include a sensor arrangement 470 including other optional sensors for monitoring physiological signals, such as an activity sensor, pressure sensor, oxygen sensor, accelerometer, microphone, piezoelectric sensor, impedance sensor, temperature sensor, and/or other sensor used to monitor a patient. These may also be provided to electrical circuitry 400 for processing.

Electrical circuitry 400 may similarly control monitoring time intervals and sampling rates according to a particular clinical application. In addition, electrical circuitry may include state machines, application specific hardware, or other sequential logic circuitry and/or integrated circuits to control device functions and need not be implemented exclusively as a microprocessor. For example, electrical circuitry 400 may include timers utilized to detect asystole events as described in more detail below.

Electrical circuitry 400 communicates with integrated antenna 322 (shown in FIG. 3A) or other communication to transmit electrical signal data, e.g. EGM signal data, stored in memory or received from electrical circuitry 400 in real time. Antenna 322 may be configured to transmit and receive communication signals via inductive coupling, electromagnetic coupling, tissue conductance, Near Field Communication (NFC), Radio Frequency Identification (RFID), BLUETOOTH®, WiFi, or other proprietary or non-proprietary wireless telemetry communication schemes.

The electrical circuitry 400 may include a communication module including the integrated antenna 322, so as to enable the insertable cardiac monitor 300 to communicate with one or more external devices located external to the device 300. For example, as shown in FIG. 4, a cardiac monitoring system 500 may include an insertable cardiac monitor 10 (e.g., insertable cardiac monitor 300), which may include a communication module for communicating with a programmer 510. The programmer 510 may include a user interface that presents information to and receives input from a user. In some variations, the programmer 510 may include, for example, a suitable computing device such as a tablet, a smartphone, desktop computer, laptop computer, and/or the like. It should be noted that the user may also interact with programmer remotely via a networked computing device. As further shown in FIG. 4, in some variations, the insertable cardiac monitor 10 and/or the programmer 510 may be configured to transfer and/or receive information (e.g., cardiac data, such as EGM data and/or cardiac episode-related information derived from the EGM data) to and/or from a secondary memory storage device 520, such as over a wired or wireless network.

A user, such as a physician, technician, surgeon, electrophysiologist, other clinician, or patient, interacts with programmer to communicate with insertable cardiac monitor 300. For example, the user may interact with programmer to retrieve physiological or diagnostic information from the insertable cardiac monitor 300. A user may also interact with programmer to program the insertable cardiac monitor 300, e.g., select values for operational parameters of the insertable cardiac monitor 300. For example, the user may use programmer to retrieve information from the insertable cardiac monitor 300 regarding the rhythm of a patient heart, trends therein over time, or arrhythmic episodes. In some variations, alerts regarding device status (e.g., health state) and/or regarding type(s) of cardiac episode(s) detection may be provided to the patient or a clinician through the programmer 510, though they may be provided in any suitable manner (e.g., personal smartphone, other computing device, pushed through to an electronic medical record, etc.). The insertable cardiac monitor 300 and the programmer may communicate via wireless communication using any techniques known in the art.

In some variations, the insertable cardiac monitor 300 (which is an example of insertable cardiac monitor 10) can be placed subcutaneously in a patient near or over the patient's heart. For example, in some variations the insertable cardiac monitor 300 can be placed in a subcutaneous pocket located over an intercostal space (e.g., over the 4th intercostal space), and positioned at a desirable angle and/or displacement relative to the patient's sternum (e.g., between about 0 and 45 degrees relative to the sternum, about 2 cm from the left edge of the sternum). Once inserted, the insertable cardiac monitor 300 may go through suitable setup and/or calibration processes.

FIG. 5 is a conceptual perspective schematic diagram of an implantable medical device 500 (IMD 500), according to various examples described in this disclosure. IMD 500 may be a leadless, subcutaneously implantable monitoring device including a proximal electrode 502A located at proximal end 504, a distal electrode 502B located at distal end 506 (collectively “electrodes 502”), a housing 508, electrical circuitry, an optical sensor arrangement 510 (comprising, for example optical sensor(s)), an integrated antenna 512, and a power source. In particular, electrical circuitry is coupled to proximal electrode 502B and distal electrode 502A to sense cardiac signals and monitor events. Electrical circuitry may also be connected to transmit and receive communications via integrated antenna 512. The power source can provide power to electrical circuitry, as well as to any other components that require power. The power source may include one or more energy storage devices, such as one or more rechargeable or non-rechargeable batteries. In some examples, electrical circuitry includes processing circuitry and a storage device, such as memory (e.g., as shown in FIG. 3B), the memory being operatively coupled to the processing circuitry and configured to store data and/or instructions.

Electrical circuitry may be configured to receive one or more electrophysiological signals such as an electromyography (EMG) signal, an electrocardiography (ECG) signal, an electroencephalography (EEG) signal, and/or any biopotential-based signal. In the example shown in FIG. 5, electrical circuitry may receive one or more electrophysiological signals (e.g., raw EGM or EMG signals) monitored by the proximal electrode 502A and distal electrode 502B and raw optical signals monitored by the optical sensor arrangement 510. Electrical circuitry may include components/modules for converting the raw EGM signal to a processed EGM signal that can be analyzed to detect sense events and for converting the raw optical signals to calibrated processed optical signal(s) that can be analyzed to detect sense events. Although not shown, electrical circuitry may include any discrete and/or integrated electronic circuit components that implement analog and/or digital circuits capable of producing the functions described for analyzing optical signal(s) to determine a health condition status of a patient. For example, the electrical circuitry may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, a processor (shared, dedicated, or group) that executes one or more software or firmware programs, memory devices, or any other suitable components or combination thereof that provide the described functionality.

In one example, electrical circuitry includes a sensing unit for monitoring the EGM signal detected by the respective proximal 502A and distal electrodes 502B and light signals received by the optical sensor arrangement 510, respectively. In one example, electrical circuitry includes processing circuitry that is utilized to receive information regarding sensed events and implements one or more algorithms for determining a health condition status of a patient. In addition, the analog voltage signals received from the electrodes 502 may be passed to analog-to-digital (A/D) converters included in the electrical circuitry and stored in a memory unit included as part of electrical circuitry for subsequent analysis with firmware executed by the processor included as part of electrical circuitry.

Some variations of the IMD 500 (e.g., examples shown in FIGS. 3A, 3B, and 5) include a container 514 and an insulative cover 516. In some examples, the insulative cover 516 may include an optical window, the optical window transparent to at least a portion of a visible light spectrum and/or a near-visible light spectrum (e.g., infrared light or ultraviolet light). In some examples, the optical window comprises an optically clear material such as sapphire, glass, and/or plastic. In some examples, the optical window may be formed of the same material as insulative cover 516. In some examples, the optical window may be a portion of insulative cover 516. In some examples, the optical window comprises the entire insulative cover 516. In some examples, the insulative cover 516 is optically transparent (e.g., the insulative cover 516 comprises transparent sapphire). The proximal electrode 502A and the distal electrode 502B may be formed or placed on an outer surface of cover 516. The electrical circuitry may be formed or placed on an inner surface of cover 516, or within container 514. In some examples, the antenna 512 is formed or placed on the inner surface of cover 516. In other examples, antenna 512 is formed or placed on the outer surface of cover 516, and in other examples, antenna 512 may be formed or placed at least partially on the inner surface and partially on the outer surface of cover 516. In some examples, insulative cover 516 may be positioned over an open container 15 such that container 514 and cover 516 form housing 20 and enclose electrical circuitry (and in some cases antenna 512) and protect the circuitries from fluids such as body fluids. For example, the housing 508 may be a hermetically-sealed housing configured for subcutaneous implantation within a patient, wherein at least the power source, memory, and processing circuitry are within the hermetically-sealed case, and in some examples, the optical sensor arrangement 510 are within the hermetically-sealed case.

The electrical circuitry may be formed on the inner side of insulative cover 516, such as by using flip-chip or wire bond integrated circuit packaging technology. Insulative cover 516 may be flipped onto a container 514. When flipped and placed onto container 514, the components of IMD 500 formed on the inner side of insulative cover 516 may be positioned in a gap defined by container 514. The electrodes 502 and the antenna 512 (when placed or formed on the outer surface of cover 516) may be electrically connected to sensing circuitry and communication circuitry, respectively, e.g., through one or more vias formed through insulative cover 516. The insulative cover 516 may be formed of sapphire (i.e., corundum), glass, and/or any other suitable insulating material. The container 514 may be formed from any suitable material configured to house electrical circuitry, support and mate with cover 516 to isolate electrical circuitry from contact with tissue and/or fluids of a patient, and to be implantable within the patient. In some examples, container 514 may house power source (e.g., a battery). In some examples, container 514 may also be electrically conductive. For example, container 514 may be formed from titanium or any other suitable material (e.g., a biocompatible material). Electrodes 502 may be formed from any of stainless steel, titanium, platinum, iridium, or alloys thereof. In addition, electrodes 502 may be coated with a material such as titanium nitride or fractal titanium nitride, although other suitable materials and coatings for such electrodes may be used.

In some variations, the IMD 500 is defined by a length L, a width W and thickness or depth D and is in the form of an elongated rectangular prism wherein the length L is much larger than the width W, which in turn is larger than the depth D, as illustrated in FIG. 5. In one example, the geometry of the IMD 500—in particular a width W greater than the depth D—is selected to allow the IMD 500 to be inserted under the skin of the patient using a minimally invasive procedure and to remain in the desired orientation during insert. For example, the IMD 500 may include a radial asymmetry (notably, a rectangular shape) along the longitudinal axis that maintains the device in the proper orientation following insertion. For example, in one example the spacing between the proximal electrode 502A and the distal electrode 502B may range from 30 millimeters (mm) to 55 mm, 35 mm to 55 mm, and from 40 mm to 55 mm and may be any range or individual spacing from 25 mm to 60 mm. In another example the spacing between the proximal electrode 502A and the distal electrode 502B may range from 15 mm to 30 mm, 17 mm to 28 mm, and from 20 mm to 28 mm and may be any range or individual spacing from 12 mm to 30 mm. In addition, the IMD 500 may have a length L that ranges from 30 mm to about 70 mm. In other variations, the length L may range from 40 mm to 60 mm, 45 mm to 60 mm and may be any length or range of lengths between about 30 mm and about 70 mm. In some examples, the IMD 500 may have a length L that ranges from 15 mm to about 35 mm, or from 20 mm to 30 mm, 22 mm to 30 mm and may be any length or range of lengths between about 15 mm and about 35 mm. In addition, the width W of a major surface of the IMD 500, e.g., insulative cover 516 in the example shown in FIG. 5, may range from 3 mm to 10 mm and may be any single or range of widths between 3 mm and 10 mm, or may range from 1.5 mm to 5 mm and may be any single or range of width between 1.5 mm and 5 mm. The thickness of depth D of the IMD 500 may range from 2 mm to 9 mm, or from 1.5 mm to 4.5 mm. In other variations, the depth D of the IMD 500 may range from 2 mm to 5 mm and may be any single or range of depths from 2 mm to 9 mm, or may range from 1 mm to 2.5 mm and may be any single or range of depths from 1 mm to 4.5 mm. In addition, IMD 500 according to an example of the present invention has a geometry and size designed for ease of implant and patient comfort. Examples of the IMD 500 described in this disclosure may have a volume of 3 cubic centimeters (cm) or less, 1.5 cubic cm or less or any volume between 3 and 1.5 cubic centimeters, or may have a volume of 1.5 cubic centimeters (cm) or less, 0.75 cubic cm or less or any volume between 1.5 and 0.75 cubic centimeters.

In some variations, the implantable medical device 10 can be placed subcutaneously in a patient near or over the patient's heart. For example, in some variations the implantable medical device 10 can be placed in a subcutaneous pocket located over an intercostal space (e.g., over the 4^{th} intercostal space), and positioned at a desirable angle and/or displacement relative to the patient's sternum (e.g., between about 0 and 45 degrees relative to the sternum, about 2 cm from the left edge of the sternum). Once inserted, the implantable medical device 300 may go through suitable setup and/or calibration processes.

In some examples, the implantable medical device 10 is implanted outside of a thoracic cavity of a patient (e.g., subcutaneously in a pectoral location). The implantable medical device 10 may be positioned near the sternum near or just below the level of the heart of the patient, e.g., at least partially within the cardiac silhouette. In some variations, the implantable medical device 10 includes a plurality of electrodes and is configured to sense a cardiac electrogram (EGM) via the plurality of electrodes, as well as other physiological signals and/or parameters via the optical sensor arrangement 360. In some examples, the implantable medical device 10 takes the form of an insertable cardiac monitor (ICM) such as the RevealLINQ™ or LINQ II™ ICM, or other ICM similar to, e.g., a version or modification of the RevealLINQ™ or LINQ II™ ICM. Although described primarily in the context of examples in which implantable medical device 10 is an ICM, in various examples, the implantable medical device 10 may represent a cardiac monitor, a defibrillator, a cardiac resynchronization pacer/defibrillator, a pacemaker, an implantable pressure sensor, a neurostimulator, or any other implantable or external medical device.

Depiction of different features as modules is intended to highlight different functional aspects and does not necessarily imply that such modules must be realized by separate hardware or software components. Rather, functionality associated with one or more modules may be performed by separate hardware, firmware and/or software components, or integrated within common hardware, firmware and/or software components.

Furthermore, it should be understood that the systems and methods described herein in accordance with the present technology are not limited to the implantable medical devices 300, 500 described herein with respect to FIGS. 3A, 3B and 5. Rather, the systems and methods described herein in accordance with the present technology may additionally or alternatively be used in conjunction with other implantable medical device, in conjunction with other cardiac monitoring devices (e.g., other leadless cardiac monitoring devices, cardiac monitoring devices with leads, etc.), and/or in conjunction with other optical sensing devices (e.g., a wearable device measuring PPG).

### II. Methods for Assessment of Patient Conditions

Generally, methods in accordance with the present technology may include assessing one or more patient conditions using sounds that are generated within the body of a patient and detected with an implantable device (and/or an external device such as a wearable device). For example, body sounds may be detected with an implantable device such as a cardiac monitoring device (e.g., insertable cardiac monitor 300, 500) or other suitable implantable monitoring device including a sensor arrangement with one or more sound sensors. Specifically, cardiovascular conditions (e.g., issues relating to native and/or artificial heart valves, vascular occlusions, abdominal aortic aneurysms, deep vein thrombosis, hemorrhage, peripheral vascular disease, ischemic stroke, heart failure exacerbation, and/or chronic obstructive pulmonary disease (COPD)) may be detected, monitored, and/or otherwise assessed based on the body sounds from the patient.

Any of the analysis of body sounds can be performed using one or more software algorithms, such as rule-based algorithms, machine learning algorithms, or combinations thereof. In some variations, the body sounds may be filtered (e.g., to remove signal noise) using any suitable analog and/or digital techniques (e.g., one or more suitable software algorithms), such as prior to analysis. In some variations, such characterization of cardiovascular condition(s) may be performed at least in part with an artificial intelligence model, such as a pre-trained machine learning model (e.g., classification-type model, such as a neural network or other deep learning model), though it may additionally or alternatively performed with any suitable algorithm. Examples of machine learning algorithms that may be used include: clustering algorithms, regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning), regularization algorithms (e.g., ridge regression, least absolute shrinkage and selection operator, elastic net, least-angle regression), decision tree algorithms (e.g., Iterative Dichotomiser 3 (ID3), C4.5, C5.0, classification and regression trees, chi-squared automatic interaction detection, decision stump, M5), Bayesian algorithms (e.g., naïve Bayes, Gaussian naïve Bayes, multinomial naïve Bayes, averaged one-dependence estimators, Bayesian belief networks, Bayesian networks, hidden Markov models, conditional random fields), clustering algorithms (e.g., k-means, single-linkage clustering, k-medians, expectation maximization, hierarchical clustering, fuzzy clustering, density-based spatial clustering of applications with noise (DBSCAN), ordering points to identify cluster structure (OPTICS), non negative matrix factorization (NMF), latent Dirichlet allocation (LDA), Gaussian mixture model (GMM)), association rule learning algorithms (e.g., apriori algorithm, equivalent class transformation (Eclat) algorithm, frequent pattern (FP) growth), artificial neural network algorithms (e.g., perceptrons, neural networks, back-propagation, Hopfield networks, autoencoders, Boltzmann machines, restricted Boltzmann machines, spiking neural nets, radial basis function networks), deep learning algorithms (e.g., deep Boltzmann machines, deep belief networks, convolutional neural networks, stacked auto-encoders), dimensionality reduction algorithms (e.g., principle component analysis (PCA), independent component analysis (ICA), principle component regression (PCR), partial least squares regression (PLSR), Sammon mapping, multidimensional scaling, projection pursuit, linear discriminant analysis, mixture discriminant analysis, quadratic discriminant analysis, flexible discriminant analysis), ensemble algorithms (e.g., boosting, bootstrapped aggregation, AdaBoost, blending, gradient boosting machines, gradient boosted regression trees, random forest), or suitable combinations thereof. The machine learning algorithms described herein can be trained using any suitable technique, including supervised learning, unsupervised learning, semi-supervised learning, reinforcement learning, or suitable combinations thereof.

In some variations, a machine learning algorithm may be pre-trained on patient-centric training data; that is, trained based on using a patient's existing body sounds signature as a baseline. Additionally or alternatively, in some variations a machine learning algorithm may be pre-trained on data relating to one or more known patient characteristics (e.g., medical conditions, demographic such as age, sex, weight, BMI, etc.); for example, in some variations the machine learning algorithm may be pre-trained using training data derived from patients known to have mitral regurgitation, and the pre-trained machine learning algorithm may be used to analyze body sounds for various patients suspected of having, or potentially having, mitral regurgitation. Statistical analysis can be performed to detect what changes in the body sound signal may be different and/or unique for each patient. Accordingly, the software algorithm(s) for analyzing body sounds can be tailored for each individual patient being monitored.

Examples of such methods for assessing a patient condition are described in further detail below.

### A. Valvular Dysfunction

Cardiac murmurs can yield information on myocardial and valvular dysfunction. Murmurs are abnormal sounds and are produced by one of more valve conditions including (i) backward regurgitation through a leaky valve, (ii) forward flow through a narrow or deformed valve, (iii) turbulent flow through a normal or abnormal valve, or (iv) vibration of loose structures within the heart. Based on the time of occurrence, murmurs are typically classified into systolic murmurs and diastolic murmurs. Systolic and diastolic murmurs can be analyzed to help diagnose, monitor, and/or otherwise assess valvular conditions in a patient, as further described herein.

FIG. 1 is a schematic flowchart of an example method 100 for assessing a patient condition based on sounds. The method 100 includes receiving a sensor signal indicative of heart sounds of a patient 110, and characterizing a heart valve condition of the patient based on the sensor signal 120, such as by use of an artificial intelligence model (e.g., machine learning model). In some variations, the sensor signal is obtained with a sensor arrangement including one or more heart sound sensors (e.g., accelerometer, microphone, piezoelectric crystal element) and/or other concurrent sensors, where the sensor arrangement is part of an implantable device. The implantable device may include multiple functionalities. For example, in some variations the implantable device may be a cardiac monitoring device (e.g., insertable cardiac monitor 300, 500) or other suitable implantable monitoring device including a sensor arrangement with one or more sound sensors. Furthermore, in some variations at least a portion of the sensor arrangement may be arranged in an external device, such as a wearable device.

In some variations, method 100 may enable continuous monitoring performance of a heart valve (e.g., native heart valve such as a mitral valve, aortic valve, or tricuspid valve, or a prosthetic valve). For example, method 100 may be used to detect and/or diagnose presence of a heart valve condition, such as mitral valve regurgitation, mitral valve stenosis, aortic valve regurgitation, and/or aortic valve stenosis. Additionally or alternatively, method 100 may be used to track progression of a heart valve condition such as any of the above. Furthermore, in some variations the method 100 may be used to perform post-surgical monitoring after valve replacement with a prosthetic valve (e.g., transcatheter aortic valve replacement (TAVR)), which can, for example, assess and track prosthetic valve performance and/or the need for further medical intervention.

Receiving a sensor signal indicative of heart sounds (e.g., process 110) functions to obtain heart sound data that may be analyzed, such as for presence and/or degree of valvular dysfunction. The heart sound sensor signal may, for example, include heart sounds such as S1, S2, S3, and S4 heart sounds, as well as mechanical sounds such as clicks, rubs, whooshes, etc.

Characterizing a heart valve condition of the patient based on the sensor signal (e.g., process 120) functions to analyze the heart sound signal, such as for presence and/or degree of valvular dysfunction. As described above, systolic and diastolic murmurs and their associated acoustic features can be analyzed to characterize a heart valve condition.

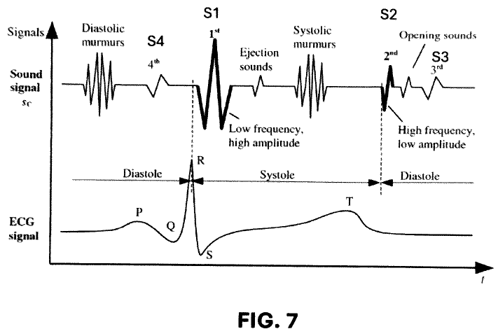

As shown in FIG. 7, systolic murmurs occur during systole, and between S1 and S2 heart sounds. Analysis of systolic murmurs can help in the assessment of various conditions. For example, an early systolic murmur (occurring relatively early in systole, such as at S1 and peaking within the first approximately 25% of systole) can be analyzed to characterize mitral regurgitation, tricuspid regurgitation, and/or ventricular septal defect. A mid-systolic murmur (occurring during a middle portion of systole, such as after S1 and before S2, and may be characterized by an increasing-decreasing or crescendo-decrescendo pattern) can be analyzed to characterize aortic valve stenosis, an issue with prosthetic aortic valve (e.g., thrombosus, regurgitation, other regurgitation), and/or a pulmonic outflow obstruction. A late systolic murmur (occurring relatively late in systole, such as with a peak toward the end of systole right before S2) can be analyzed to characterize mitral valve prolapse, tricuspid valve prolapse, and/or mitral regurgitation. A holosystolic murmur (starting right after S1 and continuing until S2) can be analyzed to characterize mitral regurgitation, tricuspid regurgitation, and/or ventricular septal defect. In some variations, timing of the murmur, in combination with a feature such as amplitude, frequency, and/or pattern of murmurs, may further help to differentiate between types of systolic murmurs.

Furthermore, as shown in FIG. 7, diastolic murmurs occur during diastole, and between S2 and the next S1 heart sounds. Analysis of diastolic murmurs can help in the assessment of various conditions. For example, an early diastolic murmur (occurring relatively early in diastole, such as at S2 with the close of the aortic and pulmonary valves and typically ending before S1) can be analyzed to characterize aortic regurgitation, pulmonic regurgitation, and/or left descending coronary artery stenosis. A mid-diastolic murmur (occurring during a middle portion of diastole, such as after S2 and before S1) can be analyzed to characterize mitral stenosis, an issue with a prosthetic mitral valve (e.g., thrombosus, regurgitation, other regurgitation), tricuspid stenosis, atrial myxoma (obstruction), and/or increased flow across the atrioventricular valve. A late diastolic murmur (occurring relatively late in diastole, such as presystolic starting after S2 and extending up to S1 with an increasing or crescendo configuration) can be analyzed to characterize mitral stenosis and/or tricuspid stenosis. In some variations, timing of the murmur, in combination with a feature such as amplitude, frequency, and/or pattern of murmurs, may further help to differentiate between types of diastolic murmurs.

The analysis of the sensor signal may be performed with one or more suitable algorithms. For example, as described above, the sensor signal indicative of heart sounds may be analyzed with a suitable artificial intelligence model or other software algorithm, as described above. In general, the software algorithm(s) for analyzing the sensor signal may, for example, evaluate the timing (e.g., a determination of whether a detected murmur is a systolic murmur or a diastolic murmur, based on onset, end, and/or duration of the murmur) and/or frequency of the signal.

Generally, murmur frequencies range from about 20 Hz to about 600 Hz. For example, mitral diastolic murmurs fall in the range of between about 20 Hz-115 Hz, and aortic diastolic murmurs occur at higher frequencies in the range of between about 140 Hz-600 Hz. The diastolic murmur containing the highest frequency sound is aortic regurgitation, whose dominant frequencies are approximately 400 Hz. The early systolic murmurs such as mitral regurgitation lay below 140 Hz but may contain components up to 400 Hz. The principal frequencies of other sounds and murmurs are between about 100 and about 400 Hz. Examples of sensor signal features and possible associated heart valve conditions are shown in Table 1 below.

TABLE 1
Example heart sound features and associated heart valve conditions

| | Heart Sound/Example Heart |
|---|---|
| Time/Frequency | Valve Condition |
| mid-late diastole cycle/20 Hz-115 Hz | diastolic murmur/mitral stenosis |
| early diastolic cycle/140 Hz-600 Hz, | diastolic murmur/aortic |
| spectral peak around 400 Hz | regurgitation |
| early systole cycle/<140 Hz | systolic murmur/mitral |
| | regurgitation |
| throughout systole cycle/dominant | systolic murmur/aortic stenosis |
| peak around 300 Hz | |

Additionally or alternatively, the energy of the S1 heart sound signal may be correlated to mitral valve functionality by the software algorithm(s). For example, reduced energy levels of the S1 heart sound may be correlated to, or be indicative of, mitral regurgitation.

Further relationships between other features and heart valve conditions may be learned and/or applied by one or more suitable pre-trained machine learning models, such as those described elsewhere herein. For example, the pre-trained machine learning model may be trained in a supervised learning or unsupervised learning process. The machine learning model may be configured to identify dominant patterns in heart sounds. Additionally or alternatively, principal component analysis (PCA) may be used to identify one or more suitable features in the heart sounds signal and identify the presence of murmurs in one of the principal components.

In some variations, the method 100 may further include analyzing concurrent sensor data from the sensor arrangement. For example, the method 100 may further include receiving concurrent sensor data from one or more additional sensors (e.g., from sensor arrangement 370, 470). Such concurrent sensor data may be used to help increase specificity in the analysis of the heart sound data and/or help provide confirmation of an analysis of the heart sound data, as described in further detail below. For example, an ECG signal may be used to aid feature extraction from the heart sound sensor signal, such as by aiding alignment between the heart sound sensor signal and the patient's cardiac cycle. As another example, blood pressure (e.g., measured with optical sensors in the sensor arrangement via PPG) may be used to confirm the analysis of certain conditions, such as aortic stenosis. As yet another example, impedance changes (e.g., measured with electrodes in the implantable device) may be indicative of fluid retention and/or blood volume overload, which may, for example, help differentiate between valvular dysfunction symptoms from heart failure symptoms. As yet another example, posture changes (e.g., measured using accelerometer data) may be measured concurrently with other observed heart sound data (e.g., valvular dysfunction, arrhythmia, etc.).

Additionally or alternatively, in some variations the analysis of sensor data may incorporate a feedback loop that is personalized to the patient. As such, the software algorithm may be dynamic and change over time. For example, in response to knowledge about the patient condition at the outset or over time, certain sensor signals may have heightened importance. For example, if a patient is known to have a prosthetic valve, the software algorithm(s) may put more analytical weight on sounds that are known to be associated with a prosthetic valve (e.g., loud, high-frequency, and/or metallic closing sound. As another example, in response to detecting a significant change (e.g., a certain threshold percentage change in a feature such as energy level of the heart sound signal), the software algorithm may incorporate one or more additional sensor modalities (e.g., blood pressure, ECG, impedance, other concurrent secondary sensor data such as that described herein) to help provide more sensitivity and/or confidence in the analysis of the heart valve condition.

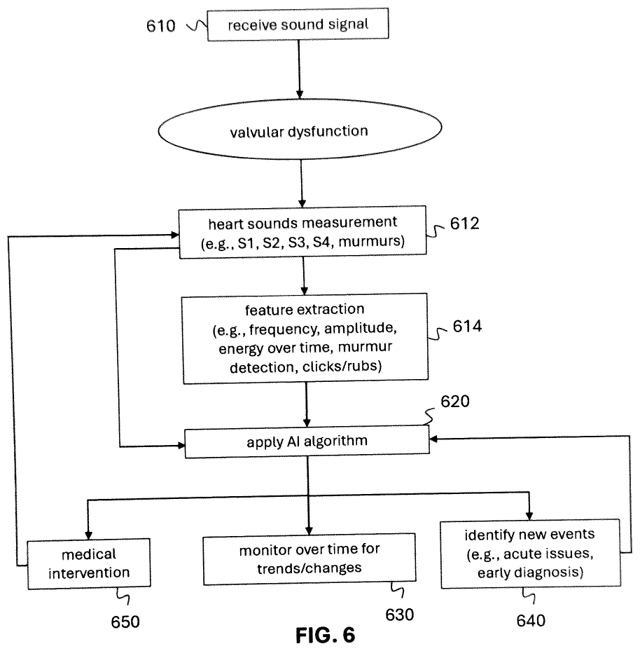

FIG. 6 is a detailed schematic of an example implementation of method 100. As shown in FIG. 6, a sensor signal indicative of heart sounds for a patient is received in block 610. The patient may have valvular dysfunction, which can be reflected in certain heart sounds as referenced in block 612. For example, one or more features may be extracted from the heart sound signal, such as frequency, amplitude, timing (e.g., relative to S1, S2, S3, and/or S4 heart sounds), energy of the signal over time, and/or the like. Types of sound effects, such as abnormal sounds indicative of murmurs, or clicks, rubs, whooshes, etc. may additionally or alternatively be identified in the heart sound signal. An artificial intelligence algorithm may help identify and/or be applied to analyze such features in block 620. Additionally or alternatively, the same or another artificial intelligence algorithm may be applied to analyze the raw heart sounds signal (and/or a filtered version thereof). These processes can be repeated over time, such as continuously, periodically/intermittently (e.g., every five seconds, every minute, every five minutes, every thirty minutes, every hour, every five hours, twice a day, etc.), triggered in response to patient status (e.g., in response to an indication that the patient is still and/or sedentary, as detected by an accelerometer for example), and/or triggered in response to a manual instruction to detect and analyze heart sounds. As such, in some variations a valvular dysfunction may be monitored over time for any trends or changes (e.g., progression of valvular disease) in block 630. Additionally or alternatively, new valvular dysfunction may be identified in block 640. One or both chronic and acute valvular dysfunction may warrant medical intervention in block 650 to address the dysfunction (e.g., implantation of a prosthetic valve, pharmaceutical treatments, etc.).

### B. Peripheral Vasculature Conditions

A variety of peripheral vasculature conditions may be assessed using body sounds, such as those sounds obtained with an implantable medical device (e.g., insertable cardiac monitor) placed proximate a region of interest (e.g., abdomen, legs). For example, sounds indicative of mechanical changes in the peripheral vasculature may be obtained with a medical device with one or more sensors positioned proximate a peripheral region of interest, and such sounds may be analyzed to detect and/or monitor aortic aneurysms, deep vein thrombosis, hemorrhage, and/or the like.

FIG. 2 is a schematic flowchart of an example method 200 for assessing a patient condition based on sounds. The method 200 includes receiving a sensor signal indicative of cardiovascular sounds of a patient 210, and characterizing a peripheral vascular condition of the patient based on the sensor signal 220, such as by use of an artificial intelligence model (e.g., machine learning model). In some variations, the sensor signal is obtained with a sensor arrangement including one or more cardiovascular sound sensors (e.g., accelerometer, microphone, piezoelectric crystal element) and/or other concurrent sensors, where the sensor arrangement is part of an implantable device. The implantable device may include multiple functionalities. For example, in some variations the implantable device may be a cardiac monitoring device (e.g., insertable cardiac monitor 300, 500) or other suitable implantable monitoring device including a sensor arrangement configured to detect cardiovascular sounds. However, in some variations the implantable device may include a sensor arrangement configured to detect solely cardiovascular sounds. The implantable device can, in some variations, be configured to be placed proximate an anatomical region of interest, such as the torso for detecting abdominal aortic aneurysms, or a leg for detecting deep vein thrombosis. Furthermore, in some variations at least a portion of the sensor arrangement may be arranged in an external device, such as a wearable device.

Receiving a sensor signal indicative of cardiovascular sounds (e.g., process 210) functions to obtain body sound data that may be analyzed, such as for presence and/or degree of one or more peripheral vasculature conditions.

Characterizing a heart valve condition of the patient based on the sensor signal (e.g., process 220) functions to analyze the cardiovascular sound signal, such as for presence and/or degree of one or more peripheral vasculature conditions. For example, in general, a hemorrhage may be characterized by a signature of frequency components associated with acoustic wave reflection, which may be captured in the cardiovascular sound signal. Furthermore, in general, a block in peripheral arteries and/or deep vein thrombosis in the legs may be characterized by a change in frequency components, a change in amplitude, and/or other features in the cardiovascular sound signal. Further relationships between other features and peripheral vasculature conditions may be learned and/or applied by a suitable pre-trained machine learning model, such as those described elsewhere herein.

The analysis of the sensor signal may be performed with one or more suitable algorithms. For example, as described above, the sensor signal indicative of cardiovascular sounds may be analyzed with a suitable artificial intelligence model or other software algorithm, as described above.

In some variations, similar to the method 100, the method 200 may further include analyzing concurrent sensor data from the sensor arrangement. For example, the method2 may further include receiving concurrent sensor data from one or more additional sensors (e.g., from sensor arrangement 370, 470). Such concurrent sensor data may be used to help increase specificity in the analysis of the cardiovascular sound data and/or help provide confirmation of an analysis of the cardiovascular sound data, as described in further detail below. For example, impedance changes (e.g., measured with electrodes in the implantable device) may be indicative of changes in peripheral vascular flow and/or edema, and may help with detection and/or other assessment of blockages in peripheral vasculature. As another example, an optical signal (e.g., PPG signal) may indicate changes in pulse transmit time changes, local tissue oxygenation, heart rate and/or heart rate variability, which may help with specificity and/or confirmation of detection and/or other assessment of blockages in peripheral vasculature (for example, an increase in pulse transit time change and/or reduction in tissue oxygenation may indicate blockages in peripheral vasculature). As yet another example, changes in temperature may be indicative of patient infection due to medical device contamination.

Additionally or alternatively, similar to method 100, in some variations the analysis of sensor data in method 200 may incorporate a feedback loop that is personalized to the patient. As such, the software algorithm may be dynamic and change over time. For example, in response to knowledge about the patient condition at the outset or over time, certain sensor signals may have heightened importance. For example, if a patient is known to have a stent graft, the software algorithm(s) may put more analytical weight on sounds that are known to be associated with the stent graft (e.g., sounds within a predetermined frequency range). As another example, in response to detecting a significant change (e.g., a certain threshold percentage change in a feature of the cardiovascular sound signal), the software algorithm may incorporate one or more additional sensor modalities (e.g., blood pressure, ECG, impedance, other concurrent secondary sensor data such as that described herein) to help provide more sensitivity and/or confidence in the analysis of the peripheral vasculature condition.

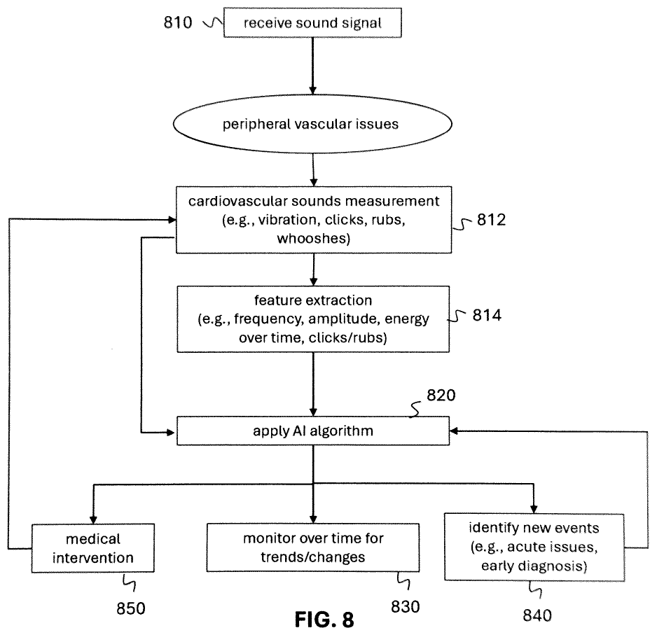

FIG. 8 is a detailed schematic of an example implementation of method 200. As shown in FIG. 8, a sensor signal indicative of cardiovascular sounds for a patient is received in block 810. The patient may have peripheral vascular issues, which can be reflected in certain cardiovascular sounds as referenced in block 812. For example, one or more features may be extracted from the cardiovascular sound signal at one or more portions of the cardiac cycle (e.g., as determined by the ECG or EGM signal), such as frequency, amplitude, timing, energy of the signal over time, and/or the like. Types of sound effects, such as clicks, rubs, whooshes, etc. may additionally or alternatively be identified in the cardiovascular sound signal. An artificial intelligence algorithm may help identify and/or be applied to analyze such features in block 820. These processes can be repeated over time, such as continuously, periodically/intermittently (e.g., every five seconds, every minute, every five minutes, every thirty minutes, every hour, every five hours, twice a day, etc.), triggered in response to patient status (e.g., in response to an indication that the patient is still and/or sedentary, as detected by an accelerometer for example), and/or triggered in response to a manual instruction to detect and analyze cardiovascular sounds. As such, in some variations a peripheral vasculature condition may be monitored over time for any trends or changes (e.g., progression of disease) in block 830. Additionally or alternatively, new peripheral vasculature conditions may be identified in block 840. One or both chronic and acute peripheral vasculature conditions may warrant medical intervention in block 850 to address the dysfunction (e.g., implantation of a stent or stent graft, pharmaceutical treatments, etc.).

### CONCLUSION

Although many of the variations are described above with respect to systems, devices, and methods for assessing heart valve and/or peripheral vasculature conditions, the technology is applicable to other applications and/or other approaches. Moreover, other variations in addition to those described herein are within the scope of the technology. Additionally, several other variations of the technology can have different configurations, components, or procedures than those described herein. A person of ordinary skill in the art, therefore, will accordingly understand that the technology can have other variations with additional elements, or the technology can have other variations without several of the features shown and described above with reference to FIGS. 1-8.

The descriptions of variations of the technology are not intended to be exhaustive or to limit the technology to the precise form disclosed above. Where the context permits, singular or plural terms may also include the plural or singular term, respectively. Although specific variations of, and examples for, the technology are described above for illustrative purposes, various equivalent modifications are possible within the scope of the technology, as those skilled in the relevant art will recognize. For example, while steps are presented in a given order, alternative variations may perform steps in a different order. The various variations described herein may also be combined to provide further variations.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific variations have been described herein for purposes of illustration, but that various modifications may be made without deviating from the technology. Further, while advantages associated with certain variations of the technology have been described in the context of those variations, other variations may also exhibit such advantages, and not all variations need necessarily exhibit such advantages to fall within the scope of the technology. Accordingly, the disclosure and associated technology can encompass other variations not expressly shown or described herein.

I/We claim:

1. A system comprising:
a sensor arrangement implantable in a patient and configured to provide a sensor signal indicative of heart sounds of the patient;
a processor; and
a memory operably coupled to the processor and storing instructions that, when executed by the processor, cause the system to perform operations comprising:
receiving the sensor signal; and
characterizing a heart valve condition of the patient by using an artificial intelligence model to analyze the sensor signal.

2. The system of claim 1, wherein the heart sounds comprise at least one of S1, S2, S3, and S4 heart sounds.

3. The system of claim 1, wherein the operations further comprise extracting one or more features of the sensor signal including at least one of frequency, amplitude, or energy over time.

4. The system of claim 1, wherein the artificial intelligence model comprises a pre-trained machine learning model.

5. The system of claim 4, wherein the pre-trained machine learning model is at least partially trained on data personalized to the patient.

6. The system of claim 1, wherein characterizing the heart valve condition comprises evaluating at least one of presence of the heart valve condition or progression of the heart valve condition over time.

7. The system of claim 1, wherein the sensor arrangement comprises at least one of an accelerometer, a microphone, or a piezoelectric crystal.

8. The system of claim 1, wherein the heart valve condition comprises at least one of stenosis or regurgitation of an aortic valve in the patient.

9. The system of claim 1, wherein the heart valve condition comprises at least one stenosis or regurgitation of a mitral valve in the patient.

10. The system of claim 1, wherein the heart valve condition comprises a status of an artificial heart valve implanted in the patient.

11. The system of claim 1, wherein the sensor arrangement, the processor, and the memory are incorporated in an implantable device.

12. The system of claim 11, wherein the implantable device comprises an insertable cardiac monitor comprising a power source and one or more electrodes.

13. The system of claim 12, wherein the insertable cardiac monitor is configured to be implanted subcutaneously in the patient.

14. The system of claim 12, wherein the insertable cardiac monitor has a volume of about 1.5 cubic centimeters or less.

15. A computer-implemented method, comprising:
receiving a sensor signal from a sensor arrangement implanted in a patient, wherein the sensor signal is indicative of heart sounds of the patient; and
characterizing a heart valve condition of the patient by using an artificial intelligence model to analyze one or more features of the sensor signal, wherein the one or more features comprises at least one of frequency, amplitude, or energy over time.

16. The method of claim 15, wherein the heart sounds comprise at least one of S1, S2, S3, and S4 heart sounds.

17. The method of claim 15, wherein the operations further comprise extracting one or more features of the sensor signal including at least one of frequency, amplitude, or energy over time.

18. The method of claim 15, wherein the artificial intelligence model comprises a pre-trained machine learning model.

19. The method of claim 18, wherein the pre-trained machine learning model is at least partially trained on data personalized to the patient.

20. The method of claim 15, wherein characterizing the heart valve condition comprises evaluating at least one of presence of the heart valve condition or progression of the heart valve condition over time.
